(19) United States
(12) Patent Application Publication
(10) Pub. No.: US 2022/0101568 A1
(43) Pub. Date: Mar. 31, 2022

(54) IMAGE GENERATION SYSTEM, IMAGE GENERATION METHOD, AND NON-TRANSITORY COMPUTER-READABLE STORAGE MEDIUM

(71) Applicant: OLYMPUS CORPORATION, Tokyo (JP)

(72) Inventors: Kota AKIYOSHI, Tokyo (JP); Tetsuya TANABE, Tokyo (JP)

(73) Assignee: OLYMPUS CORPORATION, Tokyo (JP)

(21) Appl. No.: 17/550,363

(22) Filed: Dec. 14, 2021

Related U.S. Application Data
(63) Continuation of application No. PCT/JP2019/025899, filed on Jun. 28, 2019.

(51) Int. Cl. G06T 11/00; G06T 7/00; G06T 7/90; G06T 7/62

(52) U.S. Cl. CPC G06T 11/00; G06T 7/0012; G06T 2207/30024; G06T 7/62; G06T 2207/20084; G06T 7/90

(57) ABSTRACT

An image generation system comprising a computer processor that functions as: an image input part configured to input an input image, the input image being a time-series image obtained by imaging an observed cell over time; and an image generator configured to generate a growth prediction image of the observed cell from the time-series image of the observed cell based on a first learned model, which has learned a relationship between the time-series image of a learning cell and a feature of the learning cell, and output the growth prediction image as an output image.

### CROSS-REFERENCE TO RELATED APPLICATIONS

This application is a continuation application based on a PCT Patent Application No. PCT/JP2019/025899, filed on Jun. 28, 2019, the entire content of which is hereby incorporated by reference.

### BACKGROUND

### Technical Field

The present invention relates to an image generation system for growth prediction images of cells such as microorganisms or cell-derived colonies, an image generation method, and a non-transitory computer-readable storage medium.

### Background Art

The technology for evaluating the culture state of cells such as microorganisms and cell-derived colonies has become a basic technology in a wide range of fields including advanced medical fields such as regenerative medicine and drug screening. For example, since it takes a long time for colonies of cells such as microorganisms to form colonies of a size that can be visually confirmed, a technique for evaluating colony formation at the stage of microcolonies before the colonies grow to a visible size has been developed.

Japanese Unexamined Patent Application, First Publication No. 2015-154729 (hereinafter referred to as Patent Document 1) describes a method for analyzing cells of microorganisms and the like by optical sensing. The cell analysis method described in Patent Document 1 records and analyzes an image obtained by capturing an image of an optical signal generated when cultured cells are irradiated with transmitted light over time, so that colonies that change over time can be monitored simultaneously in multiple parallels. The cell analysis method can rapidly evaluate the colonization of cells such as microorganisms, and has been conventionally carried out based on visual confirmation or microscopic observation.

However, the cell analysis method described in Patent Document 1 can monitor colonies that change over time from images recorded over time, but it has been difficult, for example, to generate a growth prediction image of colonies of cells such as microorganisms at an arbitrary designated culture elapsed time.

### SUMMARY

The present invention provides an image generation system and an image generation method capable of generating growth prediction images of cells such as microorganisms or cell-derived colonies.

An image generation system includes a computer processor that functions as: an image input part configured to input an input image, the input image being a time-series image obtained by imaging an observed cell over time; and an image generator configured to generate a growth prediction image of the observed cell from the time-series image of the observed cell based on a first learned model, which has learned a relationship between the time-series image of a learning cell and a feature of the learning cell, and output the growth prediction image as an output image.

An image generation method implemented in a computer system having a computer processor specifically programmed to perform the method includes: an input process in which an input image is input, the input image being a time-series image obtained by imaging an observed cell over time; and an image generation process in which a growth prediction image of the observed cell is generated from the time-series image of the observed cell based on a first learned model, which has learned a relationship between the time-series image of a learning cell and a feature of the learning cell, and the growth prediction image is output as an output image.

According to the image generation system and the image generation method of the present invention, it is possible to generate a growth prediction image of cells such as microorganisms or colonies derived from the cells.

### BRIEF DESCRIPTION OF THE DRAWINGS

FIG. 1 is a diagram showing a functional block of an image generation system according to a first embodiment.

FIG. 2 is a constructive conceptual diagram of a first learned model of an image generator of the image generation system.

FIG. 3 is a flowchart showing an operation of the image generation system.

FIG. 4 is a schematic diagram showing a time-lapse image input to the image generator of the image generation system and a growth prediction image to be output.

FIG. 5 is a schematic diagram showing different examples of the time-lapse image input to an image generator of the image generation system and the growth prediction image to be output.

FIG. 6 is a diagram showing a functional block of an image generation system according to a second embodiment.

FIG. 7 is a constructive conceptual diagram of a second learned model of an image determination part of the image generation system.

FIG. 8 is a flowchart showing an operation of the image generation system.

FIG. 9 is a constructive conceptual diagram of a second learned model of an image determination part of an image generation system according to a third embodiment.

FIG. 10 is a schematic diagram showing a growth prediction image input to an image generator of the image generation system and a growth prediction image output.

FIG. 11 is a flowchart showing an operation of the image generation system.

FIG. 12 is an image of cells having the same image discrimination information collected using the image generation system.

FIG. 13 shows a process of cell division to be evaluated by the image generation system.

### DETAILED DESCRIPTION OF THE PREFERRED EMBODIMENTS

### First Embodiment

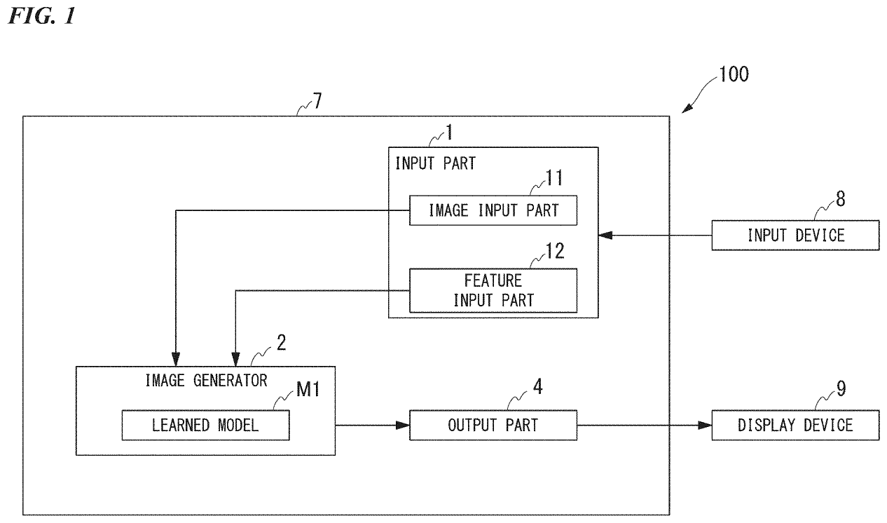

A first embodiment of the present invention will be described with reference to FIGS. 1 to 5. FIG. 1 is a diagram showing a functional block of an image generation system 100 according to the present embodiment.

### [Image Generation System 100]

The image generation system 100 includes a computer 7 capable of executing a program, an input device 8 capable of inputting data, and a display device 9 such as an LCD monitor.

The computer 7 is a program-executable device including a CPU (Central Processing Unit), a memory, a storage unit, and an input/output controller. By executing a predetermined program, it functions as a plurality of functional blocks such as the image generator 2. The computer 7 may further include a GPU (Graphics-Processing Unit), a dedicated arithmetic circuit, and the like in order to process the arithmetic executed by the image generator 2 and the like at high speed.

As shown in FIG. 1, the computer 7 includes an input part 1, an image generator 2, and an output part 4. The function of the computer 7 is realized by the computer 7 executing an image generation program provided to the computer 7.

The input part 1 receives the data input from the input device 8. The input part 1 includes an image input part II and a feature input part 12.

A time-series image obtained by capturing the observed colony X over time is input to the image input part 11. In this embodiment, the time-series image is a time-lapse image A. The time-lapse image A is a color image having a resolution of about 256 pixels in the vertical direction and 256 pixels in the horizontal direction. The time-lapse image A is a plurality of images captured over several hours to several days. The imaging interval varies depending on the observation target and is, for example, about 10 minutes for an Escherichia coli colony. The time-series image is not limited to the time-lapse image A, and may be two or more images having different shooting times.

The feature input part 12 is input with a feature (hereinafter, referred to as “designated feature D”) designated when the image generator 2 generates the growth prediction image B of the observed colony X. The feature is at least one of the elapsed culture time of the observed colony X and the size of the observed colony X. The image generation system 100 does not have to have the feature input part 12, and for example, the designated feature D can be fixedly used at a predetermined time in the culture elapsed time of the observed colony X.

The image generator 2 generates a growth prediction image B of the observed colony X corresponding to the designated feature D, from the time-lapse image A of the observed colony X input to the image input part 11, based on the “learned model (first learned model) M1”.

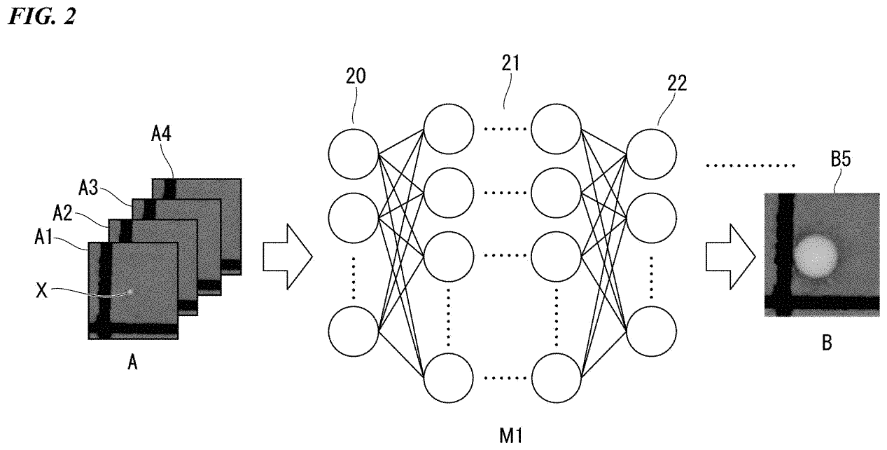

FIG. 2 is a constructive conceptual diagram of the learned model M1.

The learned model M1 is a frame prediction type deep learning model that inputs a time-lapse image A (input image) of the observed colony X input to the image input part 11, and outputs a growth prediction image B (output image) of the observed colony X corresponding to the designated feature D. The time-lapse image A of the observed colony X can be input to the learned model M1 as a plurality of input image data. The learned model M1 is implemented by, for example, PredNet (https://coxlab.github.io/prednet/), Video frame prediction by multiscale GAN (https://github.com/alokwhitewolf/Video-frame-prediction-by-multi-scale-GAN), or the like.

The learned model M1 is used as a program module of a part of the image generation program executed by the computer 7 of the image generation system 100. The computer 7 may have a dedicated logic circuit or the like for executing the learned model M1.

As shown in FIG. 2, the learned model M1 includes an input layer 20, an intermediate layer 21, and an output layer 22.

The input layer 20 receives the time-lapse image A of the observed colony X as a plurality of input images and outputs the time-lapse image A to the intermediate layer 21. When the input layer 20 receives a plurality of input images, the input layer 20 simultaneously receives the time when each input image is captured, that is, the elapsed culture time.

The intermediate layer 21 is a multi-layer neural network, and is configured by combining CNN (Convolutional Neural Network), RNN (Recurrent Neural Network), RSTM (Long short-term memory), or the like.

The output layer 22 outputs the growth prediction image B of the observed colony X corresponding to the designated feature D as an output image.

The output part 4 outputs the growth prediction image B input from the output layer 22 to the display device 9. The display device 9 displays the input growth prediction image B on an LCD monitor or the like.

[Generation of learned model M1]

The learned model M1 is generated by learning in advance the relationship between the time-lapse image of the colony and the feature of the colony. The learned model M1 may be generated by the computer 7 of the image generation system 100, or may be generated by using another computer having a higher computing power than the computer 7.

The learned model M1 is generated by a well-known technique such as backpropagation, and the filter configuration and the weighting coefficient between neurons (nodes) are updated.

In the present embodiment, the time-lapse image of the colony and the time when the colony was imaged (culture elapsed time) are the learning data. In the following description, the colony imaged for learning is referred to as a “learning colony”.

It is desirable to prepare as many learning data as possible with abundant variations regarding the types of learning colonies and the growth process. In particular, by preparing learning data of various growth processes, a learned model M1 that has high S/N discrimination ability against noise generated under various conditions and can generate a robust growth prediction image B can be generated. Specifically, it is desirable that the learning colony contain minute dust or the like that is difficult to visually distinguish from the colony.

The computer 7 generates, by supervised learning using the above-mentioned learning data, a learned model M1 in which, when the time-lapse image of the colony for learning and the designated feature D (culture elapsed time) are input into the input layer 30, an image similar to the colony growth prediction image corresponding to the input designated feature D (culture elapsed time) or the corresponding colony growth prediction image is output from the output layer 22. Further, by inputting only the time-lapse image of the learning colony to the input layer 30, a learned model M1 may be generated in which a plurality of frame prediction images are output from the output layer 22 as growth prediction images of a plurality of colonies.

### [Operation of Image Generation System 100]

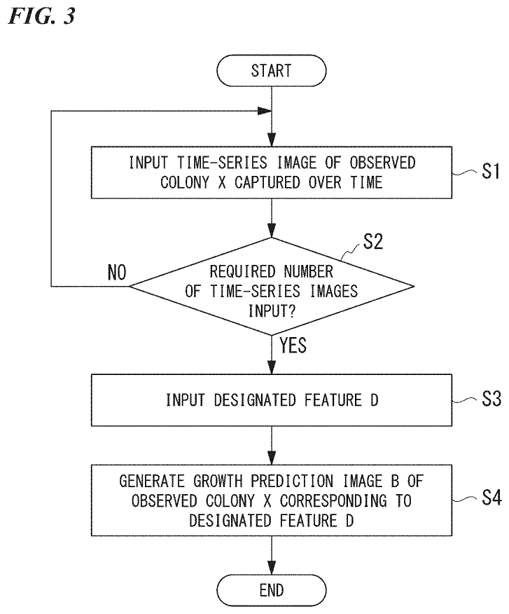

Next, the operation of the image generation system 100 will be described. FIG. 3 is a flowchart showing the operation of the image generation system 100.

A time-lapse image A obtained by capturing the observed colony X over time and a designated feature D are input to the computer 7 (input step).

Specifically, in step S1, the computer 7 accepts the input of the time-lapse image A, which is a time-series image obtained by capturing the observed colony X over time. The computer 7 determines in step S2 whether a required number of time-series images have been input. The computer 7 repeats step S1 until a required number of time-series images are input. The number of time-series images to be input is preferably large, but at least two may be sufficient.

Next, the computer 7 accepts the input of the designated feature D in step S3. Here, it is assumed that the computer 7 has input the culture elapsed time T5 as the designated feature D.

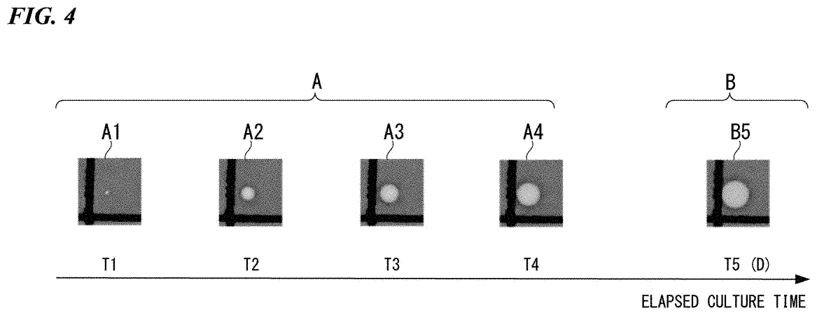

FIG. 4 is a schematic view showing a time-lapse image A input to the image generator 2 and a growth prediction image B to be output.

As shown in FIG. 4, the input time-lapse image A is composed of four images (images A1, A2, A3, A4) captured at four different culture elapsed times (culture elapsed time T1, T2, T3, T4). The time-lapse image A shown in the present embodiment is composed of only four images for the sake of simplification of the description, but the time-lapse image A actually used is generally composed of more images.

The input designated feature D is the elapsed culture time T5 of the observed colony X. The culture elapsed time T5 is longer than any of the culture elapsed times T1, T2, T3, and T4.

In step S4, the computer 7 generates a growth prediction image B5 of the observed colony X corresponding to the culture elapsed time T5 (designated feature D) of the observed colony X (image generation step). That is, the computer 7 can generate a growth prediction image B of the observed colony X after the imaging time from the input time-lapse image A.

The computer 7 outputs a growth prediction image B5 of the observed colony X corresponding to the culture elapsed time T5 (designated feature D) of the observed colony X (image output step). The display device 9 displays the input growth prediction image B5 on an LCD monitor or the like.

According to the image generation system 100 of the present embodiment, it is possible to generate a growth prediction image B of a colony of cells such as microorganisms for which a feature such as an elapsed culture time is designated. Even if minute dust or the like is contained at the stage of the micro colony before the colony grows to a visible size, the micro colony growth prediction image B can be generated by distinguishing between the minute dust or the like and the micro colony.

Although the first embodiment of the present invention has been described in detail with reference to the drawings, the specific configuration is not limited to this embodiment, and includes design changes and the like within a range that does not deviate from the gist of the present invention. In addition, the components shown in the above-described first embodiment and modified examples can be appropriately combined and configured.

### Modification Example 1

The function of the image generation system 100 may be realized by recording the image generation program in the above embodiment on a computer-readable recording medium, causing the computer system to read the program recorded on the recording medium, and executing the program. The term “computer system” as used herein includes hardware such as an OS and peripheral devices. Further, the “computer-readable recording medium” refers to a portable medium such as a flexible disk, a magneto-optical disk, a ROM, or a CD-ROM, or a storage device such as a hard disk built in a computer system. Further, a “computer-readable recording medium” may also include that which dynamically holds the program for a short period of time like a communication line for transmitting a program via a network such as the Internet or a communication line such as a telephone line, and that which holds a program for a certain period of time such as a volatile memory inside a computer system that serves as a server or a client in that case.

### Modification 2

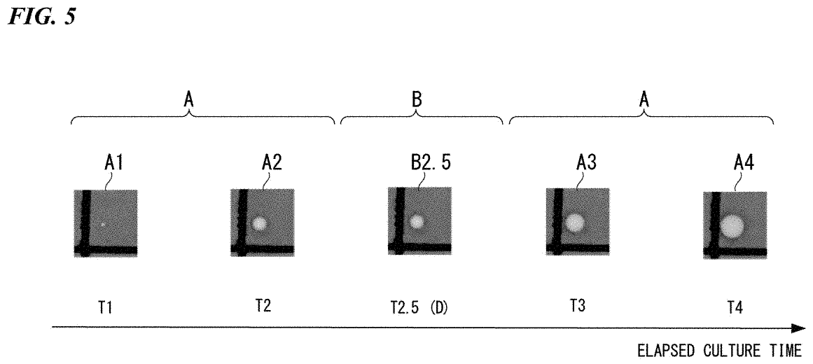

For example, in the above embodiment, the culture elapsed time T5, which has a longer culture elapsed time than any of the culture elapsed times T1, T2, T3, and T4, is designated as the designated feature D, but the culture elapsed time, which is shorter than any of the culture elapsed time T1, T2. T3, and T4, may be designated as the designated feature D. FIG. 5 is a schematic diagram showing different examples of the time-lapse image input to the image generator 2 and the output growth prediction image. The input designated feature D is the culture elapsed time T2.5, which is longer than the culture elapsed time T2 and shorter than the culture elapsed time T3. As shown in FIG. 5, the image generation system 100 generates a growth prediction image B2.5 of the observed colony X corresponding to the culture elapsed time T2.5 (designated feature D) of the observed colony X.

### Modification Example 3

For example, in the above embodiment, the time-lapse image of the observed colony X is input to the learned model M1 together with the imaging time of the image, but the mode of the learned model is not limited to this. The learned model M1 may be a model in which the cell culture conditions (temperature, nutritional state, etc.) when the time-lapse image is taken together with the time-lapse image of the observed cell O can be input together. By training the learning model by combining the cell culture conditions and the time-lapse image, the prediction accuracy of the growth prediction image is improved.

### Second Embodiment

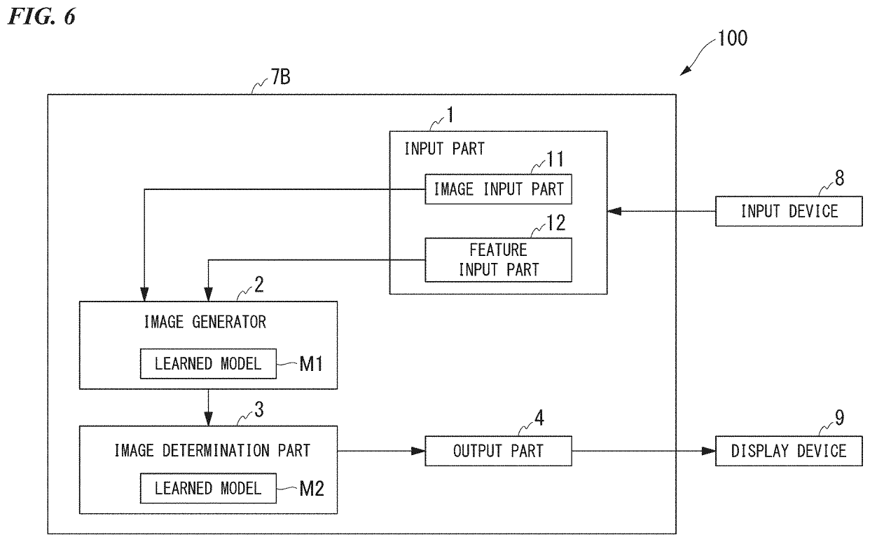

An image generation system 100B according to a second embodiment of the present invention will be described with reference to FIGS. 6 to 8. In the following description, the same reference numerals will be given to the configurations common to those already described, and duplicate description will be omitted. The image generation system 100B according to the second embodiment is different from the image generation system 100 of the first embodiment in that it further outputs image discrimination information C such as the type and state of the observed colony X.

### [Image Generation System 100B]

FIG. 6 is a diagram showing a functional block of the image generation system 100B according to the present embodiment.

The image generation system 100B includes a computer 7B capable of executing a program, an input device 8 capable of inputting data, and a display device 9 such as an LCD monitor.

The computer 7B is a program-executable device including a CPU (Central Processing Unit), a memory, a storage unit, and an input/output controller. By executing a predetermined program, it functions as a plurality of functional blocks such as the image generator 2. The computer 7B may further include a GPU (Graphics-Processing Unit), a dedicated arithmetic circuit, and the like in order to process the arithmetic executed by the image generator 2 and the like at high speed.

As shown in FIG. 6, the computer 7B includes an input part 1, an image generator 2, an image determination part 3, and an output part 4. The function of the computer 7B is realized by the computer 7B executing the image generation program provided to the computer 7B.

The image determination part 3 outputs image discrimination information C from the growth prediction image B of the observed colony X input from the image generator 2 to the image determination part 3 based on the “learned model (second learned model) M2”.

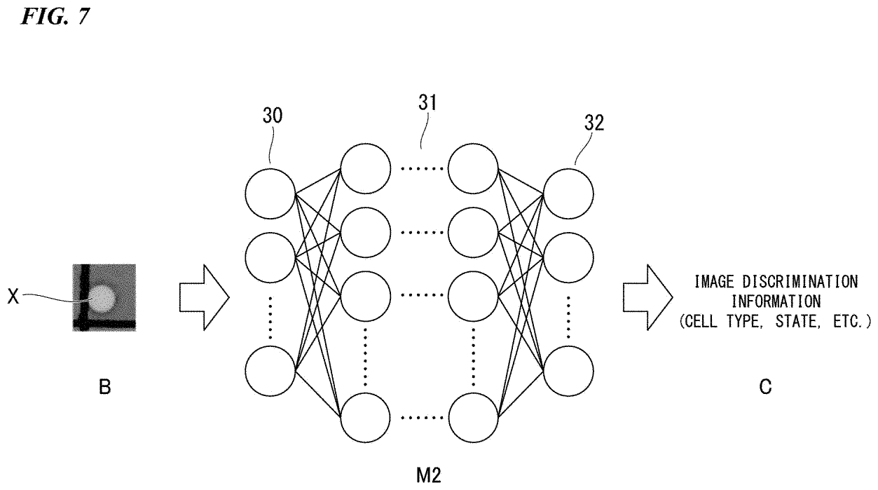

FIG. 7 is a constructive conceptual diagram of the learned model M2 of the image determination part 3.

The learned model M2 is a convolutional neural network (CNN) in which the growth prediction image B (input image) of the observed colony X is input from the image generator 2 and the image discrimination information C such as the type and state of the observed colony X is output. The growth prediction image B can be input as input image data to the learned model M2.

The learned model M2 is used as a program module of a part of the image generation program executed by the computer 7B of the image generation system 100B. The computer 7B may have a dedicated logic circuit or the like for executing the learned model M2.

As shown in FIG. 7, the learned model M2 includes an input layer 30, an intermediate layer 31, and an output layer 32.

The input layer 30 receives the growth prediction image B of the observed colony X as an input image and outputs it to the intermediate layer 31.

The intermediate layer 31 is a multi-layer neural network, and is configured by combining a filter layer, a pooling layer, a connecting layer, and the like.

The output layer 32 outputs image discrimination information C such as the type and state of the observed colony X.

### [Generation of Learned Model M2]

The learned model M2 is generated by learning in advance the relationship between the image obtained by capturing the colony and the image discrimination information such as the type and state of the colony. The learned model M2 may be generated by the computer 7B of the image generation system 100B, or may be generated by using another computer having a higher computing power than the computer 7B.

The learned model M2 is generated by supervised learning by the error back propagation method (backpropagation), which is a well-known technique, and the filter configuration of the filter layer and the weighting coefficient between neurons (nodes) are updated.

In the present embodiment, the image of the learning colony captured and the data such as the type and state of the captured learning colony are the teacher data.

It is desirable to prepare as diverse teacher data as possible by changing the type and state of learning colonies. In particular, by preparing teacher data of various types and states, the learned model M2 can be generated that has high S/N discrimination ability against noise generated under various conditions and can estimate robust image discrimination information C.

The computer 7B inputs an image of the learning colony to the input layer 30, and learns the filter configuration of the filter layer and the weighting coefficient between neurons (nodes) so that the root mean square error between the data such as the type and state of the learning colony captured by the teacher data and the image discrimination information C output from the output layer 32 becomes small.

### [Operation of Image Generation System 100B]

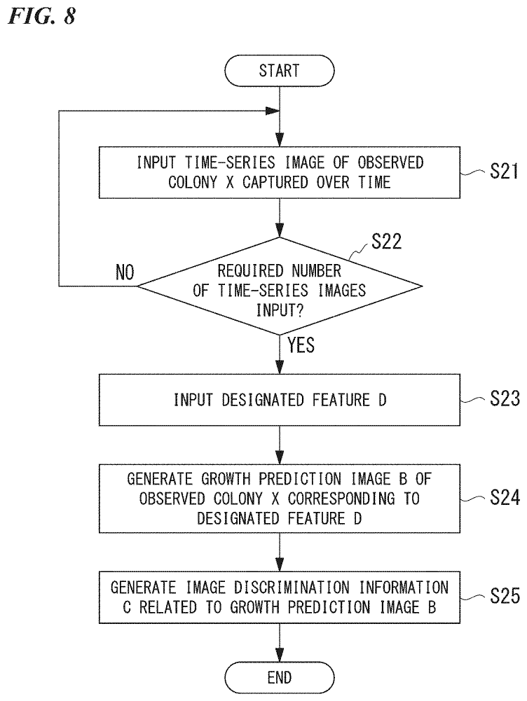

Next, the operation of the image generation system 100B will be described. FIG. 8 is a flowchart showing the operation of the image generation system 100B.

Similar to the first embodiment, the computer 7B is input with the time-lapse image A obtained by capturing the observed colony X over time and the designated feature D (input step).

Specifically, in step S21, the computer 7B accepts the input of the time-lapse image A, which is a time-series image obtained by capturing the observed colony X over time. The computer 7B determines in step S22 whether a required number of time-series images have been input. The computer 7B repeats step S21 until a required number of time-series images are input. The number of time-series images to be input is preferably large, but at least two may be sufficient.

Next, the computer 7B accepts the input of the designated feature D in step S23. Similar to the first embodiment, the image generator 2 of the computer 7B outputs the growth prediction image B of the observed colony X corresponding to the designated feature D (step S24).

In step S25, the computer 7B inputs the growth prediction image B to the image determination part 3 and generates image discrimination information C regarding the growth prediction image B (image discrimination information generation step). The display device 9 displays the input growth prediction image B and image discrimination information C on an LCD monitor or the like.

According to the image generation system 100B of the present embodiment, a growth prediction image B of a colony of cells such as microorganisms for which a feature such as an elapsed culture time is designated is generated, and further, image discrimination information C regarding the growth prediction image B can be generated. Further, the image generation system 100B can also identify the type of cells such as microorganisms from the image discrimination information C such as the generated staining result, shape, and size.

Although the second embodiment of the present invention has been described in detail with reference to the drawings, the specific configuration is not limited to this embodiment, and includes design changes and the like within a range that does not deviate from the gist of the present invention. In addition, the components shown in the above-described embodiments and modifications can be appropriately combined and configured.

### Modification Example 4

For example, in the above embodiment, the discrimination using the second learned model M2 is performed, but when the discrimination can be performed by using a conventional analyzer that does not use machine learning, the determination using the analyzer may be performed.

### Third Embodiment

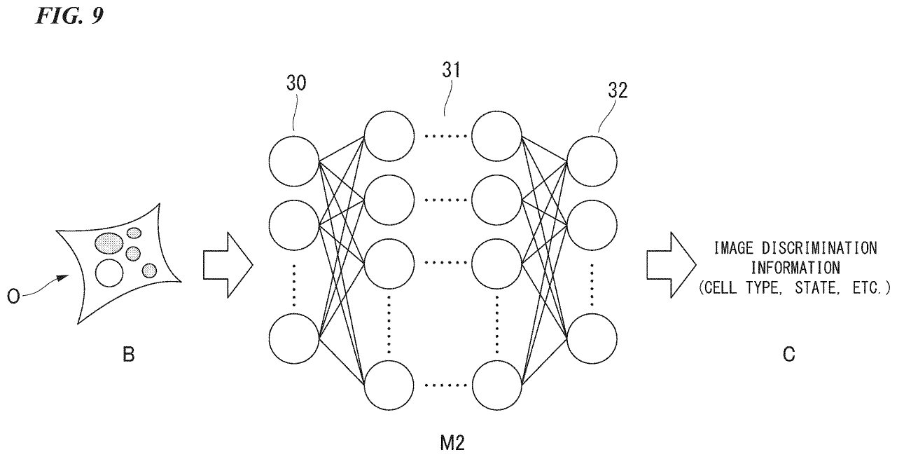

An image generator image generation system 100C according to a third embodiment of the present invention will be described with reference to FIGS. 9 to 13. In the following description, the same reference numerals will be given to the configurations common to those already described, and duplicate description will be omitted. The image generation device image generation system 100C according to the third embodiment is different from the image generation device image generation system 100B of the second embodiment in that it outputs image discrimination information C such as the type and state of the observed cell O.

### [Image Generation System 100C]

The image generation system 100C has the same configuration as the image generation system 100B according to the second embodiment. A time-lapse image A, which is a time-series image obtained by capturing the observed cells O over time instead of the observed colony X, is input to the image generation system 100C. Further, the learned model M1 of the image generation system 100C is generated by learning in advance the relationship between the time-lapse image of the learning cell and the feature of the learning cell, not the learning colony.

FIG. 9 is a constructive conceptual diagram of the learned model M2 of the image determination part 3.

The learned model M2 of the image generation system 100C is generated by learning in advance the relationship between the image obtained by capturing the learning cells instead of the learning colonies and the image discrimination information such as the type and state of the learning cells.

### [Operation of Image Generation System 100C]

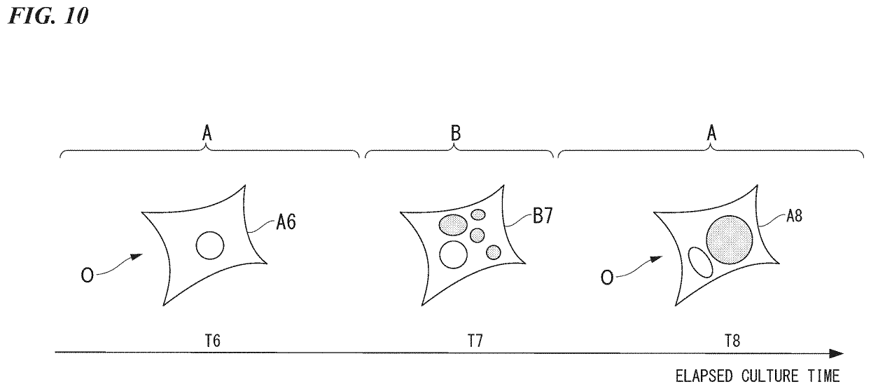

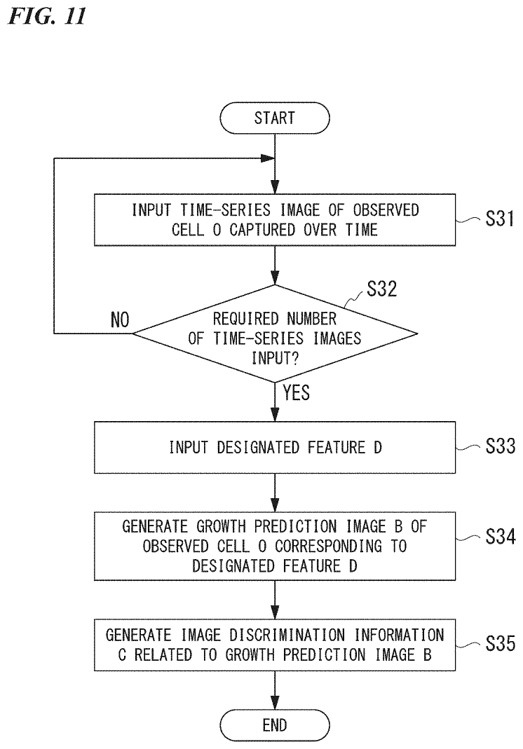

Next, the operation of the image generation system 100C will be described. FIG. 10 is a schematic view showing a time-lapse image A input to the image generator 2 and a growth prediction image B of the observed cell O to be output. FIG. 11 is a flowchart showing the operation of the image generation system 100C.

A time-lapse image A, which is an image of the observed cells O over time, and a designated feature D are input to the computer 7B (input step).

Specifically, in step S31, the computer 7B accepts the input of the time-lapse image A, which is a time-series image obtained by capturing the observed cell O over time. The computer 7 determines in step S32 whether a required number of time-series images have been input. The computer 7B repeats step S31 until a required number of time-series images are input. The number of time-series images to be input is preferably large, but at least two may be sufficient.

Next, the computer 7B accepts the input of the designated feature D in step S33. Here, it is assumed that the computer 7B inputs the culture elapsed time T7 as the designated feature D.

As shown in FIG. 10, the input time-lapse image A is composed of two images (images A6 and A8) captured at two different culture elapsed times (culture elapsed times T6 and T8), respectively. Here, image A6 is an image of “adipose progenitor cells” in adipocyte differentiation. On the other hand, image A8 is an image of “mature adipocytes” in adipocyte differentiation.

The input designated feature D is the elapsed culture time T7 of the observed cell O. The elapsed culture time T7 is longer than the elapsed culture time T6 and shorter than the elapsed culture time T8.

Similar to the first embodiment, the computer 7B generates a growth prediction image B7 of the observed cell O corresponding to the culture elapsed time T7 (designated feature D) of the observed cell O (image generation step). The generated growth prediction image B7 corresponds to an image of “immature adipocytes” in adipocyte differentiation.

In step S34, the computer 7B outputs a growth prediction image B7 of the observed cell O corresponding to the culture elapsed time T7 (designated feature D) of the observed cell O, as in the first embodiment (image output step).

In step S35, the computer 7B inputs the growth prediction image B7 to the image determination part 3 and generates the image discrimination information C regarding the growth prediction image B7 (image discrimination information generation step), as in the second embodiment. The display device 9 displays the input growth prediction image B7 and image discrimination information C on an LCD monitor or the like.

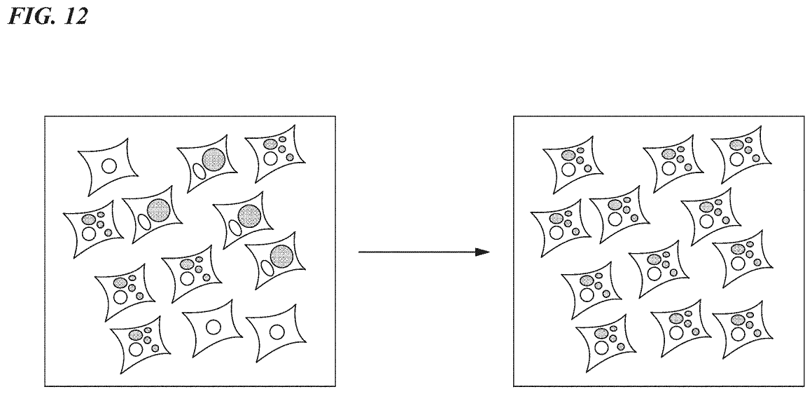

According to the image generation system 100C of the present embodiment, it is possible to generate a growth prediction image B of cells such as microorganisms for which a feature such as an elapsed culture time is designated, and further generate an image discrimination information C regarding the growth prediction image B. According to the image generation system 100C of the present embodiment, for example, a growth prediction image B having the same image discrimination information C can be collected. FIG. 12 is a collection of images of “immature adipocytes” using the image generation system 100C. The image generation system 100C can output an image of “immature adipocytes” having the same image discrimination information C, by adjusting the elapsed culture time and the like which is input as the designated feature D so that the image discrimination information C included in “immature fat cells” is output.

Although the third embodiment of the present invention has been described in detail with reference to the drawings, the specific configuration is not limited to this embodiment, and includes design changes and the like within a range that does not deviate from the gist of the present invention. In addition, the components shown in the above-described embodiments and modifications can be appropriately combined and configured.

### Modification 5

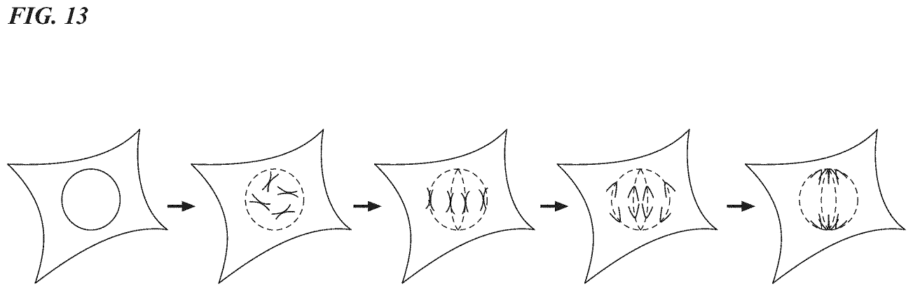

In the above embodiment, the time-lapse image A is a photograph of the course of adipocyte differentiation, and the growth prediction image B is a picture of the course of adipocyte differentiation, but the modes of the time-lapse image and the growth prediction image are not limited to this. FIG. 13 shows the course of cell division. The time-lapse image is a photograph of the course of cell division shown in FIG. 13, and the growth prediction image may be a picture of predicting the course of cell division.

### Modification 6

For example, in the above embodiment, the elapsed culture time of the observed cell O was used as the designated feature D, but the designated feature D may be the size of the observed cell O, the color of the observed cell O, the thickness of the observed cell O, the transmittance of the observed cell O, the fluorescence intensity of the observed cell O, or the luminescence intensity of observed cell O. The designated feature D may be a combination of these features.

The present invention can be applied to an image-processing device or the like that handles time-series images.

What is claimed is:

1. An image generation system comprising a computer processor that functions as:
an image input part configured to input an input image, the input image being a time-series image obtained by imaging an observed cell over time; and
an image generator configured to generate a growth prediction image of the observed cell from the time-series image of the observed cell based on a first learned model, which has learned a relationship between the time-series image of a learning cell and a feature of the learning cell, and output the growth prediction image as an output image.

2. The image generation system according to claim 1, wherein the image generator is configured to generate the growth prediction image of the observed cell corresponding to a designated feature.

3. The image generation system according to claim 1, wherein the observed cell contains a cell-derived colony.

4. The image generation system according to claim 2, wherein the feature is at least one of an elapsed culture time of the observed cell, a size of the observed cell, a color of the observed cell, a thickness of the observed cell, a transmittance of the observed cell, a fluorescence intensity of the observed cell, and a luminescence intensity of the observed cell.

5. The image generation system according to claim 1, wherein the time-series image is a time-lapse image.

6. The image generation system according to claim 1, further comprising:
an image determination part that generates image discrimination information such as a type and a state of the growth prediction image from the growth prediction image of the observed cell.

7. An image generation method implemented in a computer system having a computer processor specifically programmed to perform the method, the method comprising:
an input process in which an input image is input, the input image being a time-series image obtained by imaging an observed cell over time; and
an image generation process in which a growth prediction image of the observed cell is generated from the time-series image of the observed cell based on a first learned model, which has learned a relationship between the time-series image of a learning cell and a feature of the learning cell, and the growth prediction image is output as an output image.

8. The image generation method according to claim 7, wherein, in the image generation step, the growth prediction image of the observed cell corresponding to the designated feature is generated.

9. The image generation method according to claim 7, wherein the observed cell contains cell-derived colonies.

10. The image generation method according to claim 8, wherein the feature is at least one of an elapsed culture time of the observed cell, a size of the observed cell, a color of the observed cell, a thickness of the observed cell, a transmittance of the observed cell, a fluorescence intensity of the observed cell, and a luminescence intensity of the observed cell.

11. The image generation method according to claim 7, wherein the time-series image is a time-lapse image.

12. The image generation method according to claim 7, further comprising:
an image discrimination information generation step in which image discrimination information such as a type and a state of the growth prediction image is generated from the growth prediction image of the observed cell.

13. The image generation system according to claim 2, wherein the growth prediction image includes a figure that predicts growth of a cell reflected in the input image.

14. The image generation system according to claim 1, comprising a display device configured to display the growth prediction image.

15. The image generation system according to claim 1, wherein the growth prediction image is a division prediction image that predicts the progress of cell division.

16. The image generation system according to claim 1, wherein the growth prediction image is a differentiation prediction image that predicts the differentiation process of a cell.

17. The image generation system according to claim 4, wherein
the input image is at least two or more time-series images corresponding to different culture elapsed times Tn (where n is a natural number),
the designated feature is an elapsed culture time of the observed cell, and
the designated feature is longer than T1 having a shortest elapsed time among elapsed culture times of the two or more time-series images, and shorter than Tn which is one of the elapsed times (where T≠Tn).

18. The image generation system according to claim 6, wherein the image determination part is configured to
collect a plurality of growth prediction images having the same image discrimination information, and
output an image having the same image discrimination information of a plurality of observed cells, based on the plurality of growth prediction images.

19. A non-transitory computer-readable medium with an executable program stored thereon, wherein the program instructs a processor to perform:
an input process in which a time-series image obtained by imaging an observed cell over time is input as an input image; and
an image generation process in which a growth prediction image of the observed cell is generated as an output image from the time-series image of the observed cell, based on a first learned model, which has learned about a relationship between the time-series image of a learning cell and a feature of the learning cell.
